(19) United States
(12) Patent Application Publication
(10) Pub. No.: US 2005/0137429 A1
(43) Pub. Date: Jun. 23, 2005

(54) Methods for purification of phenol

(76) Inventors: Prashant Anil Tatake, Mumbai (IN); Umesh Krishna Hasyagar, Bangalore (IN); Pramond Shankar Kumbhar, Bangalore (IN); Bharat Singh, Bangalore (IN); John William Fulmer, Mt. Vernon, IN (US)

Correspondence Address: CANTOR COLBURN LLP, 55 GRIFFIN RD SOUTH, BLOOMFIELD, CT, 06002, US

(21) Appl. No.: 10/758,373

(22) Filed: Jan. 15, 2004

Related U.S. Application Data
(60) Provisional application No. 60/530,563, filed on Dec. 18, 2003.

(51) Int. Cl. C07C 37/68; C07C 29/74

(52) U.S. Cl. 568/810

(57) ABSTRACT

A process for producing a purified phenol stream generally includes contacting a phenol stream containing an initial concentration of hydroxyacetone and methylbenzofuran with an acidic ion exchange resin at a temperature of 50° C. to 100° C. to concurrently reduce the initial concentration of the hydroxyacetone and the methylbenzofuran in the phenol stream to produce the purified phenol stream.

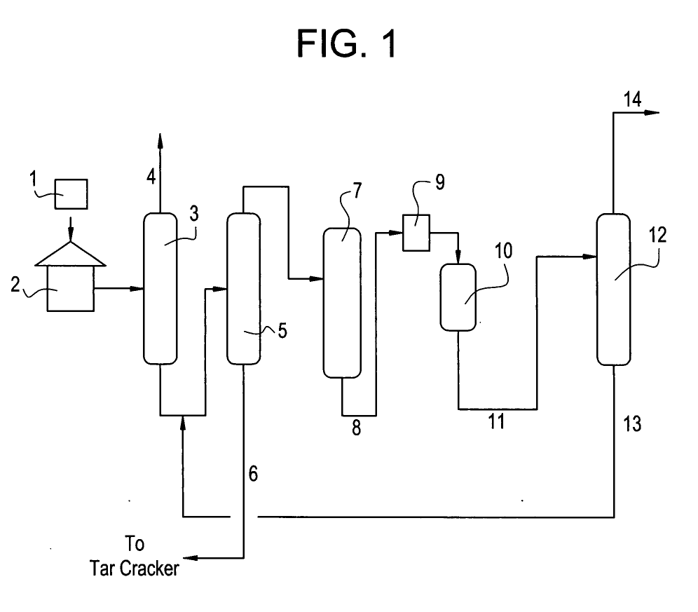

### CROSS-REFERENCE TO RELATED APPLICATIONS

This application claims priority to U.S. Provisional Application Ser. No. 60/530,563, filed Dec. 18, 2003.

### BACKGROUND

The present disclosure generally relates to a method for the purification and recovery of phenol.

A three-step cumene process generally produces about 95 percent of the phenol used in the world. Starting from benzene, the three-step cumene process involves (1) alkylation of benzene with propene to form cumene, which is typically catalyzed by phosphoric acid or aluminum chloride; (2) oxidation of cumene to cumene hydroperoxide (also referred to as “CHP”) using molecular oxygen; and (3) cleavage of CHP to phenol and acetone, which is typically catalyzed by sulfuric acid. In addition to the phenol and acetone products, unreacted cumene and various other by-products, including alpha-methylstyrene, acetophenone, cumylphenol, dimethylbenzyl alcohol, methylbenzofuran (sometimes referred to as “MBF”), and traces of various “carbonyl-type” impurities including hydroxyacetone (hereinafter also referred to herein as “acetol” and “HA”), mesityl oxide, and aldehydes are usually formed. As a result of these impurities, the resulting phenol and acetone products have to be separated from these undesirable by-products and impurities. For example, the presence of acetol and methylbenzofuran impurities in phenol renders the phenol product quality unacceptable for many end-use applications, such as in the production of bisphenol-A, diphenyl carbonate, and polycarbonate. Furthermore, phenol containing acetol and methylbenzofuran impurities tends to discolor upon aging, or during subsequent reactions, such as during sulfonation and chlorination reactions.

Depending upon the operating conditions employed, acid-catalyzed cleavage of CHP generally produces from about 1,000 to about 2,500 parts per million (ppm) of acetol. Acetol is particularly difficult to remove from phenol in the downstream process since it co-distills with phenol during rectification processes, thereby contaminating the final phenol product. Also, acetol is known to condense with phenol under the operating conditions to form methylbenzofuran(s), thereby leading to elevated equilibrium concentrations of MBF, which is/are very difficult to separate from phenol by distillation methods due to formation of an azeotrope with the phenol.

Since conventional distillation methods are not effective for removing acetol and methylbenzofuran impurities from the phenol product, various chemical treatment methods have been attempted to achieve their removal. Both homogeneous and heterogeneous processes involving basic and acidic treating agents, such as sodium hydroxide, amines, ion-exchange resins, and zeolites have been employed. Two-step techniques of passing the phenol product twice over an acid catalyst have been attempted. The two-step process generally includes initially passing the phenol over the acid catalyst at a temperature of 115° C. to reduce the initial HA concentration. However, an increase in the MBF concentration occurs at the temperatures employed, thereby requiring the second step to effect removal of MBF. Other two-step processes include first reducing acetol in the phenol product followed by an acid treatment. Such additional steps increase operational costs, which are not desirable for a high volume product like phenol.

Therefore, a continuing need exists for a more effective, single step process to reduce the level of acetol and methylbenzofuran impurities in phenol. Such a process leads to a more economical method for producing purified phenol suitable for producing higher quality aromatic bisphenols and polycarbonates.

### BRIEF SUMMARY

A one-step process for producing a purified phenol stream comprises contacting a phenol stream containing an initial concentration of hydroxyacetone and methylbenzofuran with an acidic ion exchange resin at a temperature of 50° C. to 100° C. to concurrently reduce the initial concentration of the hydroxyacetone and the methylbenzofuran in the phenol stream to produce the purified phenol stream.

In another embodiment, a continuous process for producing a purified phenol stream comprises contacting a phenol stream at a temperature of 50° C. to 100° C. at a weighted hourly space velocity of 0.1 to 5 with a sulfonated styrene-divinylbenzene acidic ion exchange resin, wherein the resin is crosslinked with greater than or equal to about 8 weight percent of divinylbenzene relative to an overall weight of said acidic ion exchange resin, and wherein the phenol stream has an initial concentration of hydroxyacetone and methylbenzofuran to concurrently reduce the initial concentration of the hydroxyacetone and methylbenzofuran and form products having a boiling point greater than phenol; and distilling said treated phenol stream.

In yet another embodiment, a process comprises contacting a phenol stream containing an initial concentration of hydroxyacetone and methylbenzofuran with an acidic ion exchange resin at a temperature of about 50° C. to about 100° C. to concurrently reduce the initial concentration of the hydroxyacetone and the methylbenzofuran in the phenol stream.

The present disclosure may be understood more readily by reference to the following detailed description of the various features of the disclosure, the figure, and the examples included therein.

### BRIEF DESCRIPTION OF THE DRAWINGS

The FIGURE is a graphical representation of a process for the production of purified phenol in accordance with the present disclosure.

### DETAILED DESCRIPTION

The FIGURE graphically illustrates a process for the production of purified phenol. Stream 1 is a crude phenol stream from a cumene to phenol process that is neutralized and stored in surge tank 2. The crude phenol is passed through distillation columns 3, 5, and 7, which generates crude acetone-stream 4, a tar-stream 6, and an impure phenol-stream 8 that comprises generally about 97 to about 99 weight percent phenol. Trace impurities from this phenol process include about 100 to about 200 ppm of HA and MBF. Stream 8 is passed continuously through a fixed-bed reactor 10 packed with ion exchange resin at a temperature of about 50° C. to about 100° C. The temperature of Stream 8 is maintained by a temperature control mechanism 9. The effluent stream 11 with reduced HA and MBF is fed to final distillation column 12 where a high boiling impurities stream 13 is separated from a purified phenol-stream 14.

By implementing the present one-step process for the reduction of impurities from phenol, it is possible to purify phenol obtained from diverse sources. Thus, in one embodiment, a suitable phenol feed 1 comprises phenol obtained from a phenol manufacturing process. In another embodiment, a suitable phenol feed 1 comprises phenol used as a raw material for other manufacturing processes to produce other polymer building blocks; non-limiting examples of which include aromatic bisphenols, such as bisphenol A; and diaryl carbonates, such as diphenyl carbonate. More particularly, the phenol feed comprises crude phenol obtained from an acid-catalyzed decomposition of CHP.

Any acidic ion exchange resin can be used as the catalyst for converting the impurities to higher boiling point compounds, i.e., a boiling point higher than phenol. As used herein the term “acidic ion exchange resin” refers to a cation exchange resin in the hydrogen form, wherein the hydrogen ions are bound to the active sites which can be removed either by dissociation in solution or by replacement with other positive ions. The active sites of the resin have different attractive strengths for different ions, and this selective attraction serves as a means for ion exchange. Non-limiting examples of suitable acidic ion exchange resins include the series of sulfonated divinylbenzene-crosslinked styrene copolymers, such as for example, copolymers crosslinked with about 1 to about 20 weight percent of divinylbenzene relative to the overall weight of the acidic ion-exchange resin. More specifically, suitable catalysts include acidic ion exchange resins crosslinked with greater than or equal to about 8 weight percent of divinylbenzene relative to the overall weight of the acidic ion exchange resin catalyst, such as for example, Amberlyst 15 commercially available from Aldrich Chemical Company, Bayer K2431 commercially available from Bayer Company, T-66 commercially available from Thermax, Ltd., and the like. Other suitable resins can be commercially obtained from producers such as, Bayer Inc., Rohm and Haas Chemical Company, Dow Chemical Company, Thermax India, Permutit and Purolite Inc. The catalysts show effective, concurrent reduction of acetol at initial concentrations of less than or equal to about 250 parts per million and methylbenzofuran in the phenol feed at initial concentrations of less than or equal to about 500 parts per million. Moreover, the catalysts are effective at relatively high flow rates, e.g., at weighted hourly space velocities (WHSV) from about 0.1 to about 5 as well as at lower temperatures, e.g., at temperatures of about 50° C. to about 100° C. The weighted hourly space velocity is the mass of feed per unit of catalyst per unit of time.

The one-step process described hereinabove can be conducted either in a batch process, or with suitable modifications as would be apparent to those skilled in the art, in a semi-continuous process or a continuous process. The treatment of the impure phenol stream can be accomplished in a fixed-bed or fluidized bed reactor, and more preferably, with the acidic ion-exchange resin in the fixed-bed reactor. In the batch mode, the one-step treatment is carried out for generally about 1.5 hours to about 23 hours. Generally, the treatment can be carried out for about 2 hours to achieve a satisfactory reduction of acetol and methylbenzofuran. In the continuous mode, the weighted hourly space velocity varies generally from about 0.1 to about 5. In a particular embodiment, the weighted hourly space velocity is about 1 to about 2. Prior to contacting the impure phenol stream with the acidic ionic exchange resin, the impure phenol stream is maintained at a temperature from about 50° C. to about 100° C. The catalyst bed is maintained at a temperature from about 50° C. to about 100° C. In a particular embodiment, the catalyst bed is maintained at a temperature of about 70° C. to about 90° C. It is advantageous to use lower operating temperatures (i.e., less than about 100° C.) with the acidic ion exchange resin catalyst since the ion exchange resins show decreased activity with time at higher temperatures. The treated phenol stream thus obtained has reduced levels of acetol, generally less than or equal to about 10 ppm; and methylbenzofuran, generally less than or equal to about 50 ppm. After exposure to the ion exchange resin, the phenol stream is fed to a thermal separation unit where the purified phenol can be separated and recovered. Thermal separation can be accomplished by using distillation techniques, or through other rectification methods.

In another embodiment, the impure phenol stream having an initial concentration of HA greater than equal to 10 ppm is treated with an ion exchange resin at a temperature generally of about 50° C. to about 100° C. to obtain a phenol stream having a reduced concentration of HA less than equal to about 10 ppm and a concentration of MBF less than equal to about 50 ppm.

The techniques described hereinabove enable the production of purified phenol by reducing HA and MBF concurrently in a single step and at lower operating temperatures. Advantageously, these techniques provide the ability to produce phenol and acetone at relatively high purity and with lower color, as well as the ability to operate the overall phenol production plant at a higher production rate, which represents a significant commercial advantage. The higher production rate is a consequence of the lower levels of MBF present in the treated phenol stream produced as described above. A lower MBF level (i.e., less than 50 ppm) in the treated phenol allows for a higher throughput in the downstream distillation operation, thereby leading to a lower cost process for producing purified phenol

Purified phenol obtained by the process may be used in the preparation of diphenylcarbonate or bisphenols. Diphenylcarbonate can be made by a variety of procedures including the phosgenation of phenol in an aqueous environment (slurry or melt) or in a solvent such as methylene chloride or by transesterification of dimethylcarbonate with phenol. A more direct procedure for making diphenylcarbonate involves the carbonylation of phenol with carbon monoxide. A transition metal catalyst such as a palladium catalyst is used in the carbonylation route often in combination with a quaternary ammonium halide as is generally known by those skilled in the art. Another procedure for making diphenylcarbonate involves the reaction between a cycloalkylene carbonate and phenol.

Bisphenols may be synthesized by a condensation reaction between phenol and a carbonyl-containing compound in the presence of an acid catalyst. Numerous types of acid catalysts have been used in this type of condensation reaction including hydrochloric acid, perchloric acid, borontrifluoride as well as solid acid catalysts including zeolites, acid clays, heteropolyacids and ion-exchange resins.

The bisphenols and diphenyl carbonates can be used to prepare bisphenol polycarbonates by methods known in the art. Suitable methods of preparing the polycarbonates include, but are not intended to be limited to, an interfacial method, wherein bisphenol and phosgene or bisphenol and diphenyl carbonate are directly reacted in a molten state to undergo ester interchange reaction; an ester interchange that is usually effected at temperatures of 250° C. or 330° C. in presence of catalysts such as organic acid salts, inorganic acid salts, oxides, hydroxides or hydrides of metals or alcoholates; and a phase boundary process under catalysis by tertiary amines, tertiary amines may also be used for the preparation of polycarbonate through the reaction of bisphenol and phosgene. Alternately, a polycarbonate can be prepared by the reaction of diphenyl carbonate and bisphenol in presence of an alkaline catalyst at high temperatures by using a melt transesterification polymerization method.

A further understanding of the techniques described above can be obtained by reference to certain specific examples that are provided herein for purposes of illustration only, and are not intended to be limiting.

### EXAMPLES

### Example 1

In this example, a continuous process for reduction of HA and MBF from a synthetic mixture comprising phenol, HA, and MBF is described.

In a continuous reactor system, a synthetic mixture of phenol, HA (209 parts per million) and MBF (9 parts per million) was passed through a Bayer K2431 ion exchange resin (5 grams; 15% cross link by divinyl benzene obtained Bayer Co.) at 90° C. at a weighted hourly space velocity (WHSV) of 1.66 and a residence time of 0.6 hours. The amount of HA and MBF in the resulting treated phenol effluent was found to be less than 6 parts per million and less than 1 parts per million, respectively, as measured by gas chromatography (GC).

### Examples 2-5

These examples describe a continuous process for concurrent reduction of acetol and methylbenzofuran from the synthetic mixture comprising phenol, HA, and MBF as described in Example 1. The synthetic mixture comprising phenol, HA and MBF was passed through a Bayer K2431 (15% cross link by divinyl benzene obtained Bayer Co.) ion exchange resin (5 grams). The parts per million of HA and MBF before and after treatment with the ion exchange resin are included the Table I below.

TABLE I
Effect of Temperature and WHSV on the reduction of HA and MBF from
phenol

| | Catalyst Bed | | | | | |
|---|---|---|---|---|---|---|
| Example | Temperature | | HA (ppm) | | MBF (ppm) | |
| No. | ° C. | WHSV | Before | After | Before | After |
| 2 | 90° C. | 1.0 | 209 | 6 | 52 | 2 |
| 3 | 90° C. | 2.2 | 209 | 6 | 52 | 6 |
| 4 | 90° C. | 3.2 | 209 | 6 | 52 | 6 |
| 5 | 70° C. | 1.0 | 234 | 6 | 34 | 25 |

### Example 6

This example provides a continuous process for reduction of HA and MBF from an actual phenol plant feed.

In a continuous reactor system, the actual phenol plant feed containing phenol, HA (215.7 parts per million), MBF (22.33 parts per million), and other carbonyl impurities was passed through a Bayer K2431 (15% cross link by divinyl benzene obtained Bayer Co.) ion exchange resin (12.5 grams) at 70° C. for a period of 57 days. Data for the first 17 days is included in the Table II below.

TABLE II
Effect of time on the removal of HA and MBF from phenol
stream

| Time | | | |
|---|---|---|---|
| (hours) | WHSV | HA (ppm) | MBF (ppm) |
| Initial | 1.60 | 215.7 | 22.33 |
| 54 | 1.57 | 0.876 | 17.81 |
| 71 | 1.56 | 0.876 | 15.67 |
| 77 | 1.58 | 0.876 | 16.24 |
| 94 | 1.54 | 0.876 | 16.52 |
| 101 | 1.64 | 0.876 | 16.74 |
| 166 | 1.61 | 0.876 | 21.02 |
| 174 | 1.56 | 0.876 | 21.80 |
| 190 | 1.61 | 0.876 | 20.14 |
| 198 | 1.57 | 0.876 | 20.37 |
| 214 | 1.57 | 0.876 | 22.38 |
| 222 | 1.59 | 0.876 | 21.62 |
| 358 | 1.54 | 0.876 | 27.72 |
| 367 | 1.54 | 0.876 | 26.88 |
| 382 | 1.61 | 0.876 | 26.94 |
| 390 | 1.56 | 0.876 | 26.85 |
| 406 | 1.60 | 0.876 | 26.42 |
| 415 | 1.61 | 0.876 | 28.39 |

It is observed that generally after about 166 minutes or greater, the MBF concentration increases. While not wanting to be bound by theory, it is believed that the active sites on the ion exchange resin have become saturated, i.e., exhausted, thereby indicating that the ion exchange resin needs to be regenerated or replaced in the above noted continuous process. In a batch process, as shown in the following Example 7, saturation is not an issue and concurrent reduction is distinctly observed over an extended period of time.

### Example 7

This example provides a batch process for reduction of HA and MBF from a synthetic mixture of phenol, HA, and MBF.

In a batch reactor system, a synthetic mixture of phenol, HA (187 ppm) and MBF (241 ppm) was treated with a sample of T-66 (8% cross link by divinylbenzene obtained from Thermax Co.) ion exchange resin (5 grams) at 90° C. in a batch mode. The results are shown in Table III below.

TABLE III

| Time | | |
|---|---|---|
| (minutes) | HA (ppm) | MBF (ppm) |
| Initial | 187.03 | 241 |
| 30 | 56.65 | 235 |
| 60 | 29.51 | 222 |
| 90 | 0.876 | 195 |
| 150 | 0.876 | 138 |
| 240 | 0.876 | 119 |
| 1,360 | 0.876 | 45 |

### Comparative Example

This example provides a prior art continuous process for reduction of HA and MBF from an actual phenol plant feed.

In a continuous reactor system, the actual phenol plant feed containing phenol, HA (211 parts per million), MBF (52 parts per million), and other carbonyl impurities was passed through a Bayer K2431 (15% cross link by divinyl benzene obtained Bayer Co.) ion exchange resin (12.5 grams). The temperature was increased to study the effect of temperature on amount of HA and MBF. The results are included in the Table IV below.

TABLE IV
Effect of Temperature >100° C. on the quantity of MBF

| Temp | Time in | HA | MBF | | | Time | HA | MBF | |
|---|---|---|---|---|---|---|---|---|---|
| (° C.) | (hours) | (ppm) | (ppm) | WHSV | Temp | (hours) | (ppm) | (ppm) | WHSV |
| | Initial | 211 | 52 | | 119 | 240 | 6 | 44 | 0.97 |
| 88 | 16 | 6 | 4 | 1.1 | 118 | 244 | 6 | 42 | 0.94 |
| 92 | 23 | 6 | 2 | 1.05 | 119 | 260 | 6 | 44 | 0.98 |
| 89 | 39 | 6 | 2 | 1.06 | 119 | 264 | 6 | 43 | 0.98 |
| 97 | 16 | 6 | 1 | 2.24 | 122 | 268 | 6 | 45 | 0.98 |
| 96 | 24 | 6 | 1 | 2.21 | 120 | 284 | 6 | 48 | 0.98 |
| 90 | 48 | 6 | 3 | 2.21 | 120 | 288 | 6 | 50 | 0.98 |
| 89 | 52 | 6 | 5 | 2.58 | 150 | 992 | 6 | 52 | 0.96 |
| 93 | 56 | 6 | 5 | 2.6 | 150 | 1068 | 6 | 63 | 2.18 |
| 92 | 104 | 6 | 6 | 2.6 | 148 | 1084 | 6 | 63 | 2.15 |
| 91 | 120 | 6 | 5 | 2.68 | 149 | 1088 | 6 | 64 | 2.28 |
| 93 | 124 | 6 | 6 | 3.58 | 149 | 1092 | 6 | 60 | 2.38 |
| 93 | 128 | 6 | 6 | 3.284 | 150 | 1132 | 6 | 67 | 2.22 |
| 92 | 172 | 6 | 6 | 3.24 | 148 | 1136 | 6 | 68 | 2.28 |
| 93 | 176 | 6 | 6 | 3.15 | 148 | 1156 | 6 | 68 | 3.32 |
| 92 | 220 | 6 | 6 | 3.27 | 148 | 1160 | 6 | 69 | 3.32 |
| 123 | 236 | 6 | 47 | 0.97 | 148 | 1164 | 6 | 68 | 3.32 |

The example demonstrates that the use of higher temperatures and lower residence times results in higher quantity of MBF. Thus, at the higher temperatures concurrent reduction of both HA and MBF is not possible.

Unexpectedly it has been observed that the use of lower temperatures in combination with a longer residence time provides concurrent removal of both HA and MBF. While not wanting to be bound by theory, it is believed that the observed phenomena may be because the higher temperatures used in prior art are not required to fully remove HA. Further, the longer residence times promotes conversion of MBF to high boiling materials, which do not co-distill with phenol and are thus easily removed.

While only certain features of the disclosure have been illustrated and described herein, many modifications and changes will occur to those skilled in the art. Therefore, it is to be understood that the appended claims are intended to cover all such modifications and changes within the true spirit of the disclosure.

1. A one-step process for producing a purified phenol stream, said one-step process comprising:
contacting a phenol stream containing an initial concentration of hydroxyacetone and methylbenzofuran with an acidic ion exchange resin at a temperature of 50° C. to 100° C. to concurrently reduce the initial concentration of the hydroxyacetone and the methylbenzofuran in the phenol stream to produce the purified phenol stream.

2. The one-step process of claim 1, wherein the initial concentration of the hydroxyacetone is less than or equal to 500 parts per million and wherein initial concentration of the methylbenzofuran is less than or equal to 250 parts per million of the phenol stream.

3. The one-step process of claim 1, wherein contacting the phenol stream with said acidic ion exchange resin is a batch method or a continuous method.

4. The one-step process of claim 4, wherein said batch method comprises contacting said phenol stream with the acidic ion exchange resin catalyst for a duration of about 1.5 hours to about 23 hours.

5. The one-step process of claim 4, wherein said continuous method comprises contacting said phenol stream with the acidic ion exchange resin catalyst at a weighted hourly space velocity of 0.1 to 5.

6. The one-step process of claim 4, wherein said continuous mode comprises contacting said phenol steam with the acidic ion exchange resin catalyst at a weighted hourly space velocity of 1 to 2.

7. A one-step purified phenol stream obtained in accordance with the method of claim 1, wherein said purified phenol stream comprises less than or equal to 50 parts per million of methylbenzofuran and less than or equal to 10 parts per million of hydroxyacetone.

8. The one-step process of claim 1, wherein contacting the phenol stream with said acidic ion exchange resin is at a temperature of about 70° C. to about 90° C.

9. The one-step process of claim 1, wherein said acidic ion exchange resin comprises a hydrogen form of a sulfonated styrene-divinylbenzene ion exchange resin.

10. The one-step process of claim 9, wherein said acidic ion exchange resin catalyst is crosslinked at about 1 to about 20 weight percent of divinylbenzene relative to an overall weight of said acidic ion exchange resin.

11. The one-step process of claim 9, wherein said acidic ion exchange resin catalyst is crosslinked with greater than or equal to about 8 weight percent of divinylbenzene relative to an overall weight of said acidic ion exchange resin catalyst.

12. A continuous process for producing a purified phenol stream, said process comprising:
contacting a phenol stream at a temperature of 50° C. to 100° C. and at a weighted hourly space velocity of 0.1 to 5 with a sulfonated styrene-divinylbenzene acidic ion exchange resin, wherein the resin is crosslinked with greater than or equal to about 8 weight percent of divinylbenzene relative to an overall weight of said acidic ion exchange resin, and wherein the phenol stream has an initial concentration of hydroxyacetone and methylbenzofuran to concurrently reduce the initial concentration of the hydroxyacetone and methylbenzofuran and form products having a boiling point greater than phenol; and
distilling said treated phenol stream.

13. The continuous process of claim 12, wherein the initial concentration of hydroxyacetone is less than or equal to about 500 parts per million and the initial concentration of methylbenzofuran is less than or equal to about 250 parts per million.

14. The continuous process of claim 12, wherein reducing the initial concentration of the hydroxyacetone and methylbenzofuran comprises lowering the initial concentration of the hydroxyacetone to less than or equal to about 10 parts per million and the initial concentration of the methylbenzofuran to less than or equal to about 50 parts per million.

15. A process comprising:
contacting a phenol stream containing an initial concentration of hydroxyacetone and methylbenzofuran with an acidic ion exchange resin at a temperature of 50° C. to 100° C. to concurrently reduce the initial concentration of the hydroxyacetone and the methylbenzofuran in the phenol stream.
